(19) United States
(12) Patent Application Publication
(10) Pub. No.: US 2018/0256717 A1
(43) Pub. Date: Sep. 13, 2018

(54) PROTEIN COMPOSITIONS AND USE THEREOF

(71) Applicant: Drug Discovery Laboratory AS, Oslo (NO)

(72) Inventors: Jo KLAVENESS, Oslo (NO); Aase Jorun KLAVENESS, Oslo (NO)

(73) Assignee: Drug Discovery Laboratory AS, Oslo (NO)

(21) Appl. No.: 15/538,697

(22) PCT Filed: Dec. 23, 2015
(86) PCT No.: PCT/IB2015/002540

§ 371 Date: Jun. 22, 2017

Related U.S. Application Data
(60) Provisional application No. 62/095,893, filed on Dec. 23, 2014.
(60) Provisional application No. 62/095,900, filed on Dec. 23, 2014.
(60) Provisional application No. 62/095,902, filed on Dec. 23, 2014.

(51) Int. Cl. A61K 39/395; A61K 47/20; A61K 47/26; A61K 9/00; A61K 9/19

(52) U.S. Cl. CPC A61K 39/39591; A61K 47/20; A61K 9/19; A61K 9/0019; A61K 47/26

(57) ABSTRACT

The present invention relates to a method for stabilization of pharmaceutical formulation of proteins. In particular, the present invention relates to compositions and methods for stabilizing proteins via addition of methanesulphonic and/or meglumine and example, free from particles and/or microbubbles.

This application claims priority to U.S. provisional patent application 62/095,902 filed Dec. 23, 2014, U.S. provisional patent application 62/095,900, filed Dec. 23, 2014, and U.S. provisional patent application 62/095,893, filed Dec. 23, 2014, each of which is incorporated herein by reference in its entirety.

### FIELD OF INVENTION

The present invention relates to a method for stabilization of pharmaceutical formulation of proteins. In particular, the present invention relates to compositions and methods for stabilizing proteins via addition of methanesulphonic and/or meglumine and, for example, free from particles and/or microbubbles.

### BACKGROUND

Protein-based biological drugs comprise a protein or a protein derivative as active pharmaceutical substance. In addition to the pharmaceutical, the drug product generally comprises different pharmaceutical additives. Most protein-based drugs are administered by injection, generally subcutanously or intravenously. All these products are sterile products. In general, the biological drugs are either in the form of a dry material or in the form of an aqueous solution. The dry substance, which might have been prepared by freeze drying, is generally dissolved or suspended in an aqueous solution prior to use. The biological drug in the form of a solution might be ready for injection or might be a concentrate that is diluted prior to administration.

One of the main problems with biological drugs is, in general, the stability. The stability of biological drugs is, in general, lower than for classical synthetic drugs; however, the stability challenges are different for each protein-based molecule. In general, protein degradation can be related to two different mechanisms: chemical reactions, for example, hydrolytic reactions, deamination reactions and oxidations; and more physical processes where proteins form dimers or oligomers and optionally form precipitates.

For general overviews of formulations of proteins and protein derivatives, see for example: B K Meyer (Ed.): Therapeutic Protein Drug Products. Practical Approaches to formulation in the Laboratory, Manufacturing, and the Clinic. (Woodhead Publishing, 2012, ISBN: 978-1-907568-18-3), Veronese, Francesco M. (Ed): PEGylated Protein Drugs: Basic Science and Clinical Applications (Birkhauser, 2009, ISBN978-3-7643-8679-5), Yatin R. Gokarn, Andrew Kosky, Eva Kras, Arnold McAuley, and Richard L. Remmele, Jr. Excipient Development for Pharmaceutical, Biotechnology, and Drug Delivery Systems, First Edition (Chapter 17) 2006, ISBN9780849327063, P. Buckel: Recombinant Protein Drugs (Springer Science & Business Media, 2001), Ajay K. Banga: Therapeutic Peptides and Proteins: Formulation, Processing, and Delivery Systems (CRC Press, 2005), Frank K. Bedu-Addo: Preformulation Development of Protein Drugs (Informa Healthcare, 2007, ISBN 9780849379529), Rodney Pearlman, Y. John Wang (Eds): Formulation, Characterization, and Stability of Protein Drugs: Case Histories (Springer, 978-0-306-45332-8, 2002), Lars Hovgaard, Sven Frokjaer, Marco van de Weert (Eds): Pharmaceutical Formulation Development of Peptides and Proteins (CRC Press, 2013), ), Gary Walsh Pharmaceutical Biotechnology. Concepts and Applications (John Wiley&Sons, 2007), Feroz Jameel and Susan Hershenson (Eds.) Formulation and Process Development Strategies for Manufacturing Biopharmaceuticals (Wiley, 2010).

The additives that are used in commercial pharmaceutical formulations of biological drugs are, for example: glycerol, m-cresol, sodium hydroxide, hydrochloric acid, zinc chloride, protamine sulfate, phenol, sodium dihydrogen phosphate, disodium hydrogen phosphate, sodium chloride, 2-amino-2-hydroxymethyl-propane-1,3-diol (TRIS), TRIS, HCl, polysorbate 20, zinc acetate, citric acid, mannitol, sodium citrate, polysorbate 80, sucrose (saccarose), human albumin, N-acetyltryptophane, sodium caprylate, L-arginine, phosphoric acid, dipotasium hydrogenphosphate, arginine HCl, histidine, trehalose, calcium chloride, glutathione, glycine, glycylglycine, methionine, urea, trisodium phosphate, leucine, isoleucine, threonine, glutamic acid, phenylalanine, polyoxamer 188, sodium sulfate, sodium caprylate, sodium N-acetyltryptophane, ethanol, propylene glycol, valine, phosphoric acid, benzyl alcohol, acetic acid, sodium acetate, lactose, disodium EDTA, DTPA, sorbitol, disodium succinate, succinic acid, ammonium acetate, polygeline, glucose, aspargine, magnesium sulfate, ammonium iron citrate, ammonium hydroxide, zinc formate, maltose, sorbitol, histidine HCl, calcium chloride, potassium dihydrogen phosphate, potassium chloride.

Typically, acids that are used in formulations and processes of biological drugs include for example hydrochloric acid, acetic acid, citric acid, phosphoric acid and amino acids. Typically, anions used in formulations and processes of biological drugs include chloride, acetate, succinate, hydrogen succinate, citrate, dihydrogen phosphate, hydrogen phosphate, phosphate, amino acid anions.

The most frequently used additives in addition to water are, sodium hydroxide, hydrochloric acid, sodium dihydrogen phosphate, disodium hydrogen phosphate, sodium chloride, polysorbate 20, citric acid, mannitol, sodium citrate, polysorbate 80, sucrose, histidine, histidine HCl, glycine, acetic acid and sodium acetate.

Based on the therapeutic value of protein based pharmaceuticals in the clinic, especially within oncology and immune related diseases, and the enormous interest in development of new pharmaceutical products based on proteins there are needs for new stable products and new methods for stabilization of protein based pharmaceuticals.

### SUMMARY

The present invention relates to a method for stabilization of pharmaceutical formulation of proteins. In particular, the present invention relates to compositions and methods for stabilizing proteins via addition of methanesulphonic and/or meglumine and for, example, free from particles and/or microbubbles.

For example, in some embodiments, the present invention provides a composition comprising a polypeptide and at least one methanesulfonic acid or salt thereof, methods of using the methanesulfonic acid containing compositions to stabilize a polypeptide, and the use of the methanesulfonic acid containing compositions to stabilize a polypeptide. In some embodiments, the polypeptide is a water soluble polypeptide. In some embodiments, the polypeptide is a pharmaceutical polypeptide. In some embodiments, the pharmaceutical polypeptide is a monoclonal antibody or a fragment thereof. In some embodiments, the polypeptide is a monoclonal antibody or fragment thereof linked to a drug (e.g., a cytotoxic agent), e.g., in an antibody-drug conjugate.

In some embodiments, the composition further comprises an aqueous liquid (e.g., a pharmaceutically acceptable carrier). In some embodiments, the composition is a dry formulation.

The present invention is not limited to particular methane sulfonic acid compounds. Examples include, but are not limited to, a pharmacologically inactive salt methanesulfonic acid, methane sulfonic acid, methane sulfonic acid histidine salt, methane sulfonic acid meglumine salt, and methane sulfonic acid TRIS salt. In some embodiments, the composition is free from or comprises a low concentrations of anions, with the exception of mesylate (e.g., a salt or ester of methanesulfonic acid). In some embodiments, the composition is free from one or more of acetate, sodium, or chloride. In some embodiments, the composition further comprises N-methylglucamine (meglumine) or 2-amino-2-hydroxymethyl-propane-1,3-diol (TRIS). In some embodiments, the polypeptide is more stable than in the absence of the methanesulfonic acid or salt thereof (e.g., exhibits reduced formation of dimers, polymers, aggregates and/or precipitates relative to the level in the absence of the methanesulfonic acid or salt thereof). In some embodiments, the composition is a pharmaceutical composition.

In some embodiments, the present invention provides a composition, comprising a polypeptide and at least one meglumine compound or meglumine salt, methods of using the meglumine containing compositions to stabilize a polypeptide, and the use of the meglumine containing compositions to stabilize a polypeptide. In some embodiments, the compositions further comprise a methanesulfonic acid or salt thereof as described above.

In some embodiments, the polypeptide is a water soluble polypeptide. In some embodiments, the polypeptide is a pharmaceutical polypeptide. In some embodiments, the pharmaceutical polypeptide is a monoclonal antibody or a fragment thereof. In some embodiments, the polypeptide is a monoclonal antibody or fragment thereof linked to a drug (e.g., a cytotoxic agent), e.g., in an antibody-drug conjugate. In some embodiments, the composition further comprises an aqueous liquid (e.g., a pharmaceutically acceptable carrier). In some embodiments, the composition is a dry formulation. The present invention is not limited to particular meglumine compounds. Examples include, but are not limited to, meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate. In some embodiments, the composition is free from cations. In some embodiments, the composition is free from sodium ions and/or chloride ions. In some embodiments, the composition further comprises one or more amino acids and/or salts thereof (e.g., histidine). In some embodiments, the polypeptide is present at a concentration of 1.5 molar or less (e.g., 0.5 M, 0.3 M, or less). In some embodiments, the polypeptide is more stable than in the absence of said meglumine (e.g., the polypeptide exhibits reduced formation of dimers, polymers, aggregates and/or precipitates relative to the level in the absence of meglumine). In some embodiments, the composition is a pharmaceutical composition.

In yet other embodiments, the present invention provides a container comprising a polypeptide and a pharmaceutically acceptable carrier (e.g., an aqueous solution comprising a polypeptide), wherein the container comprises gas phase with a total pressure below 760 mmHg (e.g., below 500 mmHg, below 400 mmHg, below 200 mmHg, below 100 mmHg, below 50 mmHg, below 20 mmHg, below 10 mmHg, or below 5 mmHg). In some embodiments, the polypeptide exhibits decreased aggregation on the surface of the liquid, decreased precipitation, and decreased levels of bubbles relative to the level in the absence of said gas. In some embodiments, the polypeptide is substantially free from particles and microbubbles. In some embodiments, the polypeptide is a pharmaceutical polypeptide (e.g., a monoclonal antibody or fragment thereof; an antibody-drug conjugate). In some embodiments, the polypeptide is present at a concentration of 10 mg/ml or higher. In some embodiments, the container is a vial. In some embodiments, the container further comprises a methanesulfonic acid or salt thereof and/or a meglumine compound.

Additional embodiments provide a container comprising a polypeptide and a pharmaceutically acceptable carrier (e.g., an aqueous solution comprising a polypeptide), wherein the container comprises a gas selected from nitrogen, argon, and helium with a total pressure below 760 mmHg (e.g., below 500 mmHg, below 400 mmHg, below 200 mmHg, below 100 mmHg, below 50 mmHg, below 20 mmHg, below 10 mmHg, or below 5 mmHg) in the head space.

Further embodiments provide a container comprising a polypeptide and a pharmaceutically acceptable carrier (e.g., an aqueous solution comprising a polypeptide), wherein the container comprises a first gas selected from, for example, carbon dioxide, perfluoropropane, perfluorobutane or sulfur hexafluoride in the head space at a partial pressure above 10 mmHg (e.g., above 20 mmHg, above 50 mmHg, above 100 mmHg, above 200 mmHg, above 400 mmHg, above 500 mmHg, or about 760 mmHg) and a second gas selected from, for example, nitrogen, argon, or helium at a partial pressure of less than 50 mmHg (e.g., less than 40 mmHg, less than 30 mmHg, less than 20 mmHg, less than 10 mmHg, less than 5 mmHg, or less than 3 mmHg).

A preferred aspect of the invention provides agents, formulations comprising the agents, and methods of manufacturing such formulations for stabilizing proteins and derivatives thereof for therapeutic or diagnostic use where said agents are methanesulfonic acid and/or pharmacologically inactive salts of methanesulfonic acid.

A preferred aspect of the invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid.

A preferred aspect of the invention provides agents, formulations comprising the agents, and methods of manufacturing such formulations for stabilizing proteins and derivatives thereof for therapeutic or diagnostic use during production where said agents are methanesulfonic acid and/or pharmacologically inactive salts of methanesulfonic acid.

A more preferred of aspect the invention provides agents, formulations comprising the agents, and methods of manufacturing such formulations for stabilizing monoclonal antibodies and derivatives thereof for therapeutic or diagnostic use where said agents are methanesulfonic acid and/or pharmacologically inactive salts of methanesulfonic acid.

A more preferred of aspect the invention provides agents, formulations comprising the agents, and methods of manufacturing such formulations for stabilizing antibody-drug conjugates for therapeutic or diagnostic use where said agents are methanesulfonic acid and/or pharmacologically inactive salts of methanesulfonic acid.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from chloride.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from chloride.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from chloride.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from chloride.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from chloride.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from chloride.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from acetate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from acetate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from acetate.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from acetate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from acetate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from acetate.

Another more preferred aspect the invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid.

Another preferred aspect the invention provides agents, formulations comprising the agents, and methods of manufacturing such formulations for stabilizing monoclonal antibodies and derivatives thereof for therapeutic or diagnostic use during production where said agents are methanesulfonic acid and/or pharmacologically inactive salts of methanesulfonic acid.

Another preferred aspect the invention provides agents, formulations comprising the agents, and methods of manufacturing such formulations for stabilizing antibody-drug conjugates and derivatives thereof for therapeutic or diagnostic use during production where said agents are methanesulfonic acid and/or pharmacologically inactive salts of methanesulfonic acid.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of a aqueous formulation.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of a aqueous formulation.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of a aqueous formulation.

Another more preferred aspect the invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid and where said formulation is essentially free from anions other than mesylate.

Another more preferred aspect the invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid and where said formulation is essentially free from anions other than mesylate.

Another more preferred aspect the invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid and where said formulation is essentially free from anions other than mesylate.

Another preferred aspect the invention provides agents, formulations comprising the agents, and methods of manufacturing such formulations for stabilizing proteins and derivatives thereof for therapeutic or diagnostic use during production where said agents are methanesulfonic acid and/or pharmacologically inactive salts of methanesulfonic acid and the environment during production is essentially free from or low on anions other than mesylate.

Another preferred aspect the invention provides agents, formulations comprising the agents, and methods of manufacturing such formulations for stabilizing monoclonal antibodies and derivatives thereof for therapeutic or diagnostic use during production where said agents are methanesulfonic acid and/or pharmacologically inactive salts of methanesulfonic acid and the environment during production is essentially free from or low on anions other than mesylate.

Another preferred aspect the invention provides agents, formulations comprising the agents, and methods of manufacturing such formulations for stabilizing antibody-drug conjugates and derivatives thereof for therapeutic or diagnostic use during production where said agents are methanesulfonic acid and/or pharmacologically inactive salts of methanesulfonic acid and the environment during production is essentially free from or low on anions other than mesylate.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from anions other than mesylate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from anions other than mesylate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of a dry formulation and the formulation is essentially free from anions other than mesylate.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from anions other than mesylate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from anions other than mesylate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid where said pharmaceutical formulation is in the form of an aqueous formulation and the formulation is essentially free from anions other than mesylate.

A preferred aspect the invention provides agents, formulations comprising the agents, and methods of manufacturing such formulations for stabilizing proteins and derivatives thereof for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS).

A preferred aspect the invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS).

A preferred aspect the invention provides agents, formulations comprising the agents, and methods of manufacturing such formulations for stabilizing proteins and derivatives thereof for therapeutic or diagnostic use during production where said agents are methanesulfonic acid and/or pharmacologically inactive salts of methanesulfonic acid together with N-methylglucamine (meglumine) or 2-amino-2-hydroxymethyl-propane-1,3-diol (TRIS).

A more preferred aspect the invention provides agents, formulations comprising the agents, and methods of manufacturing such formulations for stabilizing monoclonal antibodies and derivatives thereof for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS).

A more preferred aspect the invention provides agents, formulations comprising the agents, and methods of manufacturing such formulations for stabilizing antibody-drug conjugates and derivatives thereof for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS).

Another more preferred aspect the invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS).

Another preferred aspect the invention provides agents, formulations comprising the agents, and methods of manufacturing such formulations for stabilizing monoclonal antibodies and derivatives thereof for therapeutic or diagnostic use during production where said agents are methanesulfonic acid and/or pharmacologically inactive salts of methanesulfonic acid together with N-methylglucamine (meglumine) or 2-amino-2-hydroxymethyl-propane-1,3-diol (TRIS).

Another more preferred aspect the invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS).

Another preferred aspect the invention provides agents, formulations comprising the agents, and methods of manufacturing such formulations for stabilizing antibody-drug conjugates and derivatives thereof for therapeutic or diagnostic use during production where said agents are methanesulfonic acid and/or pharmacologically inactive salts of methanesulfonic acid together with N-methylglucamine (meglumine) or 2-amino-2-hydroxymethyl-propane-1,3-diol (TRIS).

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from chloride.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from chloride.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from chloride.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from chloride.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid w together with N-methylglucamine (meglumine) or 2-amino-2-hydroxymethyl-propane-1,3-diol (TRIS) where said pharmaceutical formulation is in the form of an aqueous formulation and the formulation is essentially free from chloride.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid w together with N-methylglucamine (meglumine) or 2-amino-2-hydroxymethyl-propane-1,3-diol (TRIS) where said pharmaceutical formulation is in the form of an aqueous formulation and the formulation is essentially free from chloride.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from acetate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from acetate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from acetate.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from acetate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from acetate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from acetate.

Another more preferred aspect the invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) and where said formulation is essentially free from anions other than mesylate.

Another more preferred aspect the invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) and where said formulation is essentially free from anions other than mesylate.

Another more preferred aspect the invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) and where said formulation is essentially free from anions other than mesylate.

Another preferred aspect the invention provides agents, formulations comprising the agents, and methods of manufacturing such formulations for stabilizing proteins and derivatives thereof for therapeutic or diagnostic use during production where said agents are methanesulfonic acid and/or pharmacologically inactive salts of methanesulfonic acid together with N-methylglucamine (meglumine) or 2-amino-2-hydroxymethyl-propane-1,3-diol (TRIS). and the environment during production is essentially free from or low on anions other than mesylate.

Another preferred aspect the invention provides agents, formulations comprising the agents, and methods of manufacturing such formulations for stabilizing monoclonal antibodies and derivatives thereof for therapeutic or diagnostic use during production where said agents are methanesulfonic acid and/or pharmacologically inactive salts of methanesulfonic acid together with N-methylglucamine (meglumine) or 2-amino-2-hydroxymethyl-propane-1,3-diol (TRIS). and the environment during production is essentially free from or low on anions other than mesylate.

Another preferred aspect the invention provides agents, formulations comprising the agents, and methods of manufacturing such formulations for stabilizing antibody-drug conjugates and derivatives thereof for therapeutic or diagnostic use during production where said agents are methanesulfonic acid and/or pharmacologically inactive salts of methanesulfonic acid together with N-methylglucamine (meglumine) or 2-amino-2-hydroxymethyl-propane-1,3-diol (TRIS). and the environment during production is essentially free from or low on anions other than mesylate.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from anions other than mesylate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from anions other than mesylate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from anions other than mesylate.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from anions other than mesylate.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from anions other than mesylate.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from anions other than mesylate.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from sodium.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from sodium.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of a dry formulation and the formulation is essentially free from sodium.

Another even more preferred aspect of the present invention provides agents, formulations comprising the agents, and methods of manufacturing such formulations for stabilizing proteins and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from sodium.

Another even more preferred aspect of the present invention provides agents, formulations comprising the agents, and methods of manufacturing such formulations for stabilizing monoclonal antibodies and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from sodium.

Another even more preferred aspect of the present invention provides agents, formulations comprising the agents, and methods of manufacturing such formulations for stabilizing antibody-drug conjugates and derivatives thereof in pharmaceutical formulations for therapeutic or diagnostic use where said agents are methanesulfonic acid and/or pharmacologically inactive salts of methanesulfonic acid together with N-methylglucamine (meglumine) or 2-amino-2-hydroxymethyl-propane-1,3-diol (TRIS) where said pharmaceutical formulation is in the form of an aqueous formulation and the formulation is essentially free from sodium.

A preferred aspect of the invention provides agents, formulations comprising the agents, and methods of manufacturing such formulations for stabilization of water-soluble proteins and derivatives thereof in formulations where said agents are meglumine and/or pharmacologically inactive salts of meglumine where said formulations and solutions are essentially free from sodium ions.

A more preferred aspect of the invention provides agents, formulations comprising the agents, and methods of manufacturing such formulations for stabilization of monoclonal antibodies and derivatives thereof in formulations where said agents are meglumine and/or pharmacologically inactive salts of meglumine where said formulations and solutions are essentially free from sodium ions.

A more preferred aspect of the invention provides agents, formulations comprising the agents, and methods of manufacturing such formulations for stabilization of antibody-drug conjugates and derivatives thereof in formulations where said agents are meglumine and/or pharmacologically inactive salts of meglumine where said formulations and solutions are essentially free from sodium ions.

A preferred aspect of the invention provides agents, formulations comprising the agents, and methods of manufacturing such formulations for stabilization of water-soluble proteins and derivatives thereof in formulations where said agents are meglumine and/or pharmacologically inactive salts of meglumine where said formulations and solutions are essentially free from other cations.

A more preferred aspect of the invention provides agents, formulations comprising the agents, and methods of manufacturing such formulations for stabilization of monoclonal antibodies and derivatives thereof in formulations where said agents are meglumine and/or pharmacologically inactive salts of meglumine where said formulations and solutions are essentially free from other cations.

A more preferred aspect of the invention provides agents, formulations comprising the agents, and methods of manufacturing such formulations for stabilization of antibody-drug conjugates and derivatives thereof in formulations where said agents are meglumine and/or pharmacologically inactive salts of meglumine where said formulations and solutions are essentially free from other cations.

A preferred aspect of the invention provides agents, formulations comprising the agents, and methods of manufacturing such formulations for stabilization of water-soluble proteins and derivatives thereof in formulations where said agents are meglumine and/or pharmacologically inactive salts of meglumine where said formulations and solutions are essentially free from chloride ions.

A more preferred aspect of the invention provides agents, formulations comprising the agents, and methods of manufacturing such formulations for stabilization of monoclonal antibodies and derivatives thereof in formulations where said agents are meglumine and/or pharmacologically inactive salts of meglumine where said formulations and solutions are essentially free from chloride ions.

A more preferred aspect of the invention provides agents, formulations comprising the agents, and methods of manufacturing such formulations for stabilization of antibody-drug conjugates and derivatives thereof in formulations where said agents are meglumine and/or pharmacologically inactive salts of meglumine where said formulations and solutions are essentially free from chloride ions.

A preferred aspect of the invention provides agents, formulations comprising the agents, and methods of manufacturing such formulations for stabilization of water-soluble proteins and derivatives thereof in formulations where said agents are meglumine and/or pharmacologically inactive salts of meglumine where said formulations and solutions are essentially free from sodium and chloride ions.

A more preferred aspect of the invention provides agents, formulations comprising the agents, and methods of manufacturing such formulations for stabilization of monoclonal antibodies and derivatives thereof in formulations where said agents are meglumine and/or pharmacologically inactive salts of meglumine where said formulations and solutions are essentially free from sodium and chloride ions.

A more preferred aspect of the invention provides agents, formulations comprising the agents, and methods of manufacturing such formulations for stabilization of antibody-drug conjugates and derivatives thereof in formulations where said agents are meglumine and/or pharmacologically inactive salts of meglumine where said formulations and solutions are essentially free from sodium and chloride ions.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine in combination with one or more surfactants where said formulations are in the form of dry formulations essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine in combination with one or more surfactants where said formulations are in the form of dry formulations essentially free from sodium.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially free from sodium.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations essentially free from sodium.

A preferred aspect of the invention relates to a method for stabilization of water-soluble proteins and derivatives thereof in formulations where said agents are meglumine and/or pharmacologically inactive salts of meglumine where said formulations are in the form of aqueous formulations essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations essentially free from sodium.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially free from chloride.

A more preferred aspect of the invention relates to a method for stabilization of monoclonal antibodies and derivatives thereof in formulations where said agents are meglumine and/or pharmacologically inactive salts of meglumine where said formulations are in the form of dry formulations essentially free from chloride.

A more preferred aspect of the invention relates to a method for stabilization of antibody-drug conjugates and derivatives thereof in formulations where said agents are meglumine and/or pharmacologically inactive salts of meglumine where said formulations are in the form of dry formulations essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially free from chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations essentially free from chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations essentially free from chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations essentially free from chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations essentially free from chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations essentially free from chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially free from sodium and chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially free from sodium and chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially free from sodium and chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially free from sodium and chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations essentially free from sodium and chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations essentially free from sodium and chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine in combination with one or more surfactants where said formulations are in the form of aqueous formulations essentially free from sodium and chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations essentially free from sodium and chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of monoclonal antibodies and derivatives thereof in-formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of dry formulations essentially where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A preferred aspect of the invention relates to agents, formulations comprising the agents, and methods of manufacturing such formulations for stabilization of water-soluble proteins and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of monoclonal antibodies and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A more preferred aspect of the invention relates to agents, formulations comprising the agents, and methods of manufacturing such formulations for stabilization of antibody-drug conjugates and derivatives thereof in formulations where said agents are meglumine and/or pharmacologically inactive salts of meglumine where said formulations are in the form of aqueous formulations where said meglumine and/or meglumine salt are selected among meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate and said formulation is essentially free from sodium and chloride.

A preferred aspect of the invention provides agents, formulations comprising the agents, and methods of manufacturing such formulations for stabilization of water-soluble proteins and derivatives thereof in formulations where said agents are meglumine and/or pharmacologically inactive salts of meglumine where said formulations and solutions are essentially free from sodium ions and comprising one or more amino acid and/or salts thereof.

A more preferred aspect of the invention provides agents, formulations comprising the agents, and methods of manufacturing such formulations for stabilization of monoclonal antibodies and derivatives thereof in formulations where said agents are meglumine and/or pharmacologically inactive salts of meglumine where said formulations and solutions are essentially free from sodium ions and comprising one or more amino acid and/or salts thereof.

A more preferred aspect of the invention provides agents, formulations comprising the agents, and methods of manufacturing such formulations for stabilization of antibody-drug conjugates and derivatives thereof in formulations where said agents are meglumine and/or pharmacologically inactive salts of meglumine where said formulations and solutions are essentially free from sodium ions and comprising one or more amino acid and/or salts thereof.

A preferred aspect of the invention provides agents, formulations comprising the agents, and methods of manufacturing such formulations for stabilization of water-soluble proteins and derivatives thereof in formulations where said agents are meglumine and/or pharmacologically inactive salts of meglumine where said formulations and solutions are essentially free from sodium ions and comprise histidine and/or salts thereof.

A more preferred aspect of the invention provides agents, formulations comprising the agents, and methods of manufacturing such formulations for stabilization of monoclonal antibodies and derivatives thereof in formulations where said agents are meglumine and/or pharmacologically inactive salts of meglumine where said formulations and solutions are essentially free from sodium ions and comprise histidine and/or salts thereof.

A more preferred aspect of the invention provides agents, formulations comprising the agents, and methods of manufacturing such formulations for stabilization of antibody-drug conjugates and derivatives thereof in formulations where said agents are meglumine and/or pharmacologically inactive salts of meglumine where said formulations and solutions are essentially free from sodium ions and comprise histidine and/or salts thereof.

Additional embodiments are described herein.

### DETAILED DESCRIPTION

The present invention relates to a method for stabilization of pharmaceutical formulation of proteins. In particular, the present invention relates to compositions and methods for stabilizing proteins via addition of methanesulphonic and/or meglumine and for, example, free from particles and/or microbubbles.

The present invention relates to methods and compositions for stabilization of pharmaceutical formulation of proteins. In some embodiments, the present invention relates to methanesulphonic containing compositions for use in the stabilization of proteins, formulations comprising such agents and a pharmaceutical protein, and methods of making and using such compositions (e.g., in diagnostic, therapeutic, research, and screening applications). In some embodiments, the present invention relates to meglumine containing compositions for use in the stabilization of proteins. In further embodiments, the present invention relates to an aqueous solution of protein free from particles and/or microbubbles where said protein solution in the container with a head space gas phase characterized by a gas phase with very low particle pressure of nitrogen, argon and helium. In some embodiments, one or more of the described methods and compositions are utilized in combination to stabilize pharmaceutical proteins. Exemplary stabilization compositions and methods are described herein.

The stabilization agents and gasses described herein find use in the stabilization and preparation of pharmaceutical proteins (e.g., monoclonal antibodies) at a high concentration of pharmaceutical protein along with increased stability. Thus, the compositions and methods described herein provide multiple advantages in time, cost, and efficiency or preparing and storing compositions comprising pharmaceutical proteins.

### Definitions

The term “water-soluble” related to protein and protein derivatives means a solubility of the protein of at least 1 mg/ml in pure water at 20 degrees centigrade, preferrably more than 2 mg/ml in pure water at 20 degrees, more preferrably more than 4 mg/ml in pure water at 20 degrees, even more preferrably more than 4 mg/ml in pure water at 20 degrees, further, even more preferrably more than 5 mg/ml in pure water at 20 degrees, most preferrably more than 10 mg/ml in pure water at 20 degrees.

The term “essentially free from” in the present document means that the mol-concentration of the actual species is less than the mol-concentration of stabilization agent in the formulation or solution, preferably less than 50% of the mol-concentration of the stabilization agent, more preferably less than 10% of the mol concentration of stabilization agent, most preferably not actively added to the formulation or solution.

### I. Stabilization Agents

In some embodiments, the present invention relates to methods for stabilization of proteins by use of comprising methanesulphonic acid and pharmaceutically acceptable salts thereof, the use of methane sulfonic acid to stabilize pharmaceutical compositions comprising proteins or derivatives thereof, especially related to reduce the formation of aggregates during storage. Another aspect of the present invention relates to use of methanesulphonic acid during production of proteins.

Another aspect of the present invention relates to pharmaceutical formulations of proteins or protein derivatives comprising methanesulphonic acid. Methanesulphonic acid can be in the form of free acid or in the form of a pharmaceutically acceptable salt. Preferably, the present compositions are low or substantially free from chloride, acetate and other anions. The most preferred compositions are also low on following ions: sodium, potassium, calcium and magnesium.

One aspect of the invention provides agents, formulations, and manufacturing methods for stabilizing proteins or protein derivatives where said agents are methanesulfonic acid and/or pharmacologically inactive salts of methanesulfonic acid.

The advantages of replacing one or more of the commonly used acids and/or anions in formulations with methanesulfonic acid and/or pharmacologically inactive salts of methanesulfonic acid include one or more of the following aspects: improved stability of the drug formulation, improved yield in a production process, reduced formation of aggregates in drug formulations, reduced formation of aggregates during the production process, reduced formation of protein precipitates in drug formulations, reduced formation of protein precipitates during the production process, improved shelf life of the formulation improved efficacy, reduced risk for immunological responses in patients.

The present invention is not limited to particular methane sulfonic acid compositions. Examples include, but are not limited to, methane sulfonic acid, methane sulfonic acid histidine salt, methane sulfonic acid meglumine salt, or methane sulfonic acid TRIS salt.

The agents for stabilizing proteins according to the present invention and for use in pharmaceutical formulations of proteins and during the production of proteins are methanesulfonic acid and/or pharmacologically inactive salts of methanesulfonic acid. Some preferred such agents are: methanesulfonic acid, meglumine mesylate, 2-amino-2-hydroxymethyl-propane-1,3-diol (TRIS) mesylate, sodium mesylate, potassium mesylate, salts between amino acids and methanesulphonic acid including are glutamin methanesulphonic acid salt, asparagine methanesulphonic acid salt, histidine methanesulphonic acid salt, serine methanesulphonic acid salt, threonine methanesulphonic acid salt, tyrosine methanesulphonic acid salt, cysteine methanesulphonic acid salt, methionine methanesulphonic acid salt, tryptophan methanesulphonic acid salt, alanine methanesulphonic acid salt, isoleucine methanesulphonic acid salt, leucine methanesulphonic acid salt, phenylalanine methanesulphonic acid salt, valine methanesulphonic acid salt, proline methanesulphonic acid salt, arginine monomethanesulphonic acid salt, arginine dimethanesulphonic acid salt, lysine arginine monomethanesulphonic acid salt, lysine arginine dimethanesulphonic acid salt, aspartic acid methanesulphonic acid salt and glutamic acid methanesulphonic acid salt, glycine methanesulphonic acid salt.

The most preferred agents are methanesulphonic acid, meglumine mesylate, 2-amino-2-hydroxymethyl-propane-1,3-diol (TRIS) mesylate and histidine methanesulphonic acid salt.

The salts can optionally be in the form of hydrates and solvates.

The mesylate salts can be prepared directly in the current formulations or solutions (in situ) from methane sulphonic acid and one or more equivalents of base.

The mesylate can be prepared from methanesulfonic acid and the corresponding base and isolated as a salt for later use using methods well known for a man skilled in the art. The preparation can take place in an aqueous media or in a non-aqueous media. The salt can be isolated by evaporation of the solvent or by filtration/centrifugation followed by drying. The amount of mesylate comprising additive to be used, according to the present invention, varies from protein to protein and from formulation to formulation. The maximum concentration of mesylate ions in a formulation, according to the present invention is less than 1.5 molar. More typical concentrations of mesylate are concentrations less than 0.15 molar.

In some embodiments, the pharmaceutical formulation comprising methanesulfonic acid and/or pharmacologically inactive salts of methanesulfonic acid is in the form of a dry formulation or an aqueous formulation

In some embodiments, formulations comprising methanesulfonic acid and/or pharmacologically inactive salts of methanesulfonic acid are essentially free from or comprising a very low concentration of one or more of chloride, acetate, sodium, or anions other than mesylate.

In some embodiments, compositions and methods further utilize methanesulfonic acid and/or pharmacologically inactive salts of methanesulfonic acid together with N-methylglucamine (meglumine) and/or 2-amino-2-hydroxymethyl-propane-1,3-diol (TRIS).

In some embodiments, the present invention provides the use of a combination of free base and the corresponding mesylate salt; for example histidine and histidine mesylate salt, TRIS and TRIS mesylate salt and methanesulphonic acid and meglumine mesylate salt. Some mixtures like this form a buffer.

In further embodiments, the stabilization agent is meglumine. In some embodiments, meglumine compositions are essentially free from sodium and preferably essentially free from other cations except meglumine cations. Meglumine can be in the form of free amine or in the form of a pharmaceutically acceptable salt. Preferably, the present compositions are also low or substantially free from chloride and acetate.

Meglumine (N-methyl-glucamine) is a an amino sugar present in some commercial pharmaceutical formulations; in ionic iodinated water-soluble X-ray contrast agents like for example acetrizoate and paramagnetic chelates as contrast agents for magnetic resonance imaging (MRI) like gadopentetate. Meglumine has also been used in formulations comprising low molecular weight synthetic acidic drug substances as ibuprofen and indomethacin.

The advantages by replacing one or more of the commonly used cations in formulations with meglumine and/or pharmacologically inactive salts of meglumine include one or more of the following aspects: improved stability of the drug formulation, improved yield in a production process, reduced formation of aggregates in drug formulations, reduced formation of aggregates during the production process, reduced formation of protein precipitates in drug formulations, reduced formation of protein precipitates during the production process, improved shelf life of the formulation improved efficacy, reduced risk for immunological responses in patients.

In some embodiments, the meglumine and/or pharmacologically inactive salts of meglumine are selected among, for example, meglumine, meglumine methanesulphonic acid salt, meglumine HCl, meglumine dihydrogen citrate, dimeglumine hydrogen citrate, trimeglumine citrate, meglumine dihydrogen phosphate, dimeglumine hydrogen phosphate or trimeglumine phosphate.

In some embodiments, compositions comprising meglumine and a pharmaceutical protein are essentially free from one or more of sodium ions, chloride ions, and cations other than meglumine.

In some embodiments, meglumine comprising compositions further comprise one or more amino acid and/or salts thereof (e.g., histidine).

The most preferred meglumine salt is meglumine methanesulphonic acid salt. The salts can optionally be in the form of hydrates and solvates.

The meglumine salts can be prepared directly in the current formulations or solutions (in situ) from meglumine and one or more equivalents of acid.

The meglumine salts can be prepared from meglumine and the corresponding acid and isolated as a salt for later use using methods well known for a man skilled in the art. The preparation can take place in an aqueous media or in a non-aqueous media. The salt can be isolated by evaporation of the solvent or by filtration/centrifugation followed by drying.

The salts can be in the form of a solid material (crystalline or amorphous) or in the form of a liquid or oil.

The amount of meglumine comprising additive to be used, according to the present invention, varies from protein to protein and from formulation to formulation. The maximum concentration of meglumine in a formulation, according to the present invention is less than 1.5 molar. More typical concentrations are less than 0.5 molar, preferrably less than 0.3 molar. The following examples illustrate various formulations of biological drugs comprising meglumine.

In some embodiments, the present invention provides a combination of free base and the corresponding salt; for example meglumine and meglumine mesylate optionally combined with other salts like for example histidine and histidine mesylate. Some of these mixtures like form buffers.

The present protein formulations comprising a stabilization agent are more stable than similar formulations without methanesulphonic acid or a pharmacologically inactive salt thereof. The “stability” is preferably related to reduced formation of dimers, polymers, aggregates and/or precipitates.

It is well known that aggregates, precipitates and other chemical or physical degradation products might result in immunological reactions or other adverse clinical effects, so an improvement of stability, in general, especially reduction in formation of dimers, oligomers, aggregates or precipitates will increase the safety of the drug due to reduction of the number and severeness of immunological related side effects and thereby improve the safety of the biological drug.

One further aspect of the present invention is therefore related to new protein formulations and solutions with improved safety characterized by the presence of a stabilization agent described herein.

Dimers, oligomers, aggregates or precipitates are not biologically active. Formation of these degradation products therefore reduces the clinical efficacy of the drug.

### II. Head Space Gas

In some embodiments, the present invention relates to a method for solvation and/or stabilization of pharmaceutical formulation of proteins where said formulation is an aqueous solution of protein free from particles and/or microbubbles where said protein solution in the container with a head space gas phase characterized by a gas phase with very low particle pressure of nitrogen, argon and helium. The head space gas might have high partial pressure of selected gases. The method can also be used during production of a protein-based pharmaceutical.

This invention further relates to stable pharmaceutical formulation of proteins where said formulation is an aqueous solution of protein free from particles and/or microbubbles there said protein solution in the container with a head space gas phase characterized by a gas phase with very low particle pressure of nitrogen, argon and helium. The head space gas might have high partial pressure of selected gases.

A gas or gas mixture in a closed container has a given pressure at a given temperature. In mixtures of gases, like for example in air, each gas has a partial pressure which is the hypothetical pressure of that gas if it alone occupied the volume of the mixtures at the same temperature. The total pressure of a gas mixture in a container is the sum of the particle pressures of the individual gases in the container. It is a linear relationship between the partial pressure of individual gases in the container and the percentage of the individual gases in the gas mixture. It is further a linear relationship between the particle pressures of individual gases and total pressure of the gas mixture. Air comprises approximately 78% nitrogen, approximately 20% oxygen, and approximately 1% argon plus minor amounts of other gases. At atmospheric pressure (760 mmHg), the partial pressure of nitrogen is approximately 593 mmHg, partial pressure of oxygen is approximately 160 mmHg and partial pressure of argon is approximately 7 mmHg. If the total pressure is reduced by 90% (some vacuum), the corresponding partial pressures of the gases are 59 mmHg, 16 mmHg and 0.7 mmHg, respectively.

The head space gas might have relative high particle pressure of selected gases. These selected gases are carbon dioxide, perfluoropropane, perfluorobutane or sulfurhexafluoride.

Regarding head space gas one of ordinary skill in art would do as suggested in Frokjaer and Hovgaard (Eds.) Pharmaceutical Formulation Development of Peptides and Proteins Yatlor&Francis (2000): “replace oxygen by nitrogen or argon during manufacturing” and “remove oxygen from the headspace of the final container” to improve protein stability against oxidative degradation.

The present invention is based on the unexpected observation that head space gas comprising high partial pressure of nitrogen, helium or argon has a negative inpact on solvation and/or stability of solutions of proteins.

The invention is further based on the unexpected observation that some selected gases in head space are preferred with regard to solvation and/or stability of protein solutions. These gases are carbon dioxide, perfluoropropane, perfluorobutane and sulfur hexafluoride.

The partial pressures of pure nitrogen, pure argon and pure helium are all 100%. The partial pressures of nitrogen and argon in air at atmospheric pressure are 590 torr and 7 torr; respectively.

Accordingly, in some embodiments, the present invention provides methods of stabilizing an aqueous solution of protein with a head space gas phase characterized by a gas phase with a low particle pressure of nitrogen, argon and helium. In some embodiments, the present invention provides methods of solubilizing a protein in an aqueous solution with a head space gas phase characterized by a gas phase with a low particle pressure of nitrogen, argon and helium.

In some embodiments, the head space gas phase comprises an air phase with a total pressure below 760 mmHg (1 atmosphere) (e.g., below 500, 400, 300, 200, 100, 50, 20, 10, or 5 mmHg), and thereby with particle pressure of nitrogen, argon and helium below the particle pressures in atmospheric air.

In some embodiments, the head space gas phase comprises nitrogen gas with a total pressure below 760 mmHg (1 atmosphere) (e.g., below 500, 400, 300, 200, 100, 50, 20, 10, or 5 mmHg).

In some embodiments, the head space gas phase comprises argon gas with a total pressure below 760 mmHg (1 atmosphere) (e.g., below 500, 400, 300, 200, 100, 50, 20, 10, or 5 mmHg).

In some embodiments, the head space gas phase comprises helium gas with a total pressure below 760 mmHg (1 atmosphere) (e.g., below 500, 400, 300, 200, 100, 50, 20, 10, or 5 mmHg).

In some embodiments, the head space gas phase comprises carbon dioxide with a partial pressure above 10 mmHg (e.g., above 20, 50, 100, 200, 500, or 760 mmHg) and less than 40 mmHg (e.g., less than 20, 10, 5, or 3 mmHg) partial pressure of any of the gases nitrogen, argon and helium.

In some embodiments, the head space gas phase comprises carbon dioxide with a partial pressure above 10 mmHg and less than 3 mmHg partial pressure of any of the gases nitrogen, argon and helium, carbon dioxide with a partial pressure above 20 mmHg and less than 5 mmHg partial pressure of any of the gases nitrogen, argon and helium, carbon dioxide with a partial pressure above 50 mmHg and less than 5 mmHg partial pressure of any of the gases nitrogen, argon and helium, carbon dioxide with a partial pressure above 100 mmHg and less than 10 mmHg partial pressure of any of the gases nitrogen, argon and helium, carbon dioxide with a partial pressure above 200 mmHg and less than 20 mmHg partial pressure of any of the gases nitrogen, argon and helium, carbon dioxide with a partial pressure above 500 mmHg and less than 40 mmHg partial pressure of any of the gases nitrogen, argon and helium, or carbon dioxide with a partial pressure around 760 mmHg and less than 40 mmHg partial pressure of any of the gases nitrogen, argon and helium.

In some embodiments, the head space gas phase comprises perfluoropropane with a partial pressure above 10 mmHg (e.g., above 20, 50, 100, 200, 500, or 760 mmHg) and less than 40 mmHg (e.g., less than 20, 10, 5, or 3 mmHg) partial pressure of any of the gases nitrogen, argon and helium.

In some embodiments, the head space gas phase comprises perfluoropropane with a partial pressure above 10 mmHg and less than 3 mmHg partial pressure of any of the gases nitrogen, argon and helium, perfluoropropane with a partial pressure above 20 mmHg and less than 5 mmHg partial pressure of any of the gases nitrogen, argon and helium, perfluoropropane with a partial pressure above 50 mmHg and less than 10 mmHg partial pressure of any of the gases nitrogen, argon and helium, perfluoropropane with a partial pressure above 100 mmHg and less than 20 mmHg partial pressure of any of the gases nitrogen, argon and helium, perfluoropropane with a partial pressure above 200 mmHg and less than 40 mmHg partial pressure of any of the gases nitrogen, argon and helium, perfluoropropane with a partial pressure above 500 mmHg and less than 40 mmHg partial pressure of any of the gases nitrogen, argon and helium, or perfluoropropane with a partial pressure around 760 mmHg and less than 40 mmHg partial pressure of any of the gases nitrogen, argon and helium.

In some embodiments, the head space gas phase comprises perfluorobutane with a partial pressure above 10 mmHg (e.g., above 20, 50, 100, 200, 500, or 760 mmHg) and less than 40 mmHg (e.g., less than 20, 10, 5, or 3 mmHg) partial pressure of any of the gases nitrogen, argon and helium.

In some embodiments, the head space gas phase comprises perfluorobutane with a partial pressure above 10 mmHg and less than 3 mmHg partial pressure of any of the gases nitrogen, argon and helium, perfluorobutane with a partial pressure above 20 mmHg and less than 5 mmHg partial pressure of any of the gases nitrogen, argon and helium, perfluorobutane with a partial pressure above 50 mmHg and less than 10 mmHg partial pressure of any of the gases nitrogen, argon and helium, perfluorobutane with a partial pressure above 100 mmHg and less than 20 mmHg partial pressure of any of the gases nitrogen, argon and helium, perfluorobutane with a partial pressure above 200 mmHg and less than 40 mmHg partial pressure of any of the gases nitrogen, argon and helium, perfluorobutane with a partial pressure above 500 mmHg and less than 40 mmHg partial pressure of any of the gases nitrogen, argon and helium, or perfluorobutane with a partial pressure around 760 mmHg and less than 40 mmHg partial pressure of any of the gases nitrogen, argon and helium.

In some embodiments, the head space gas phase comprises sulfur hexafluoride with a partial pressure above 10 mmHg (e.g., above 20, 50, 100, 200, 500, or 760 mmHg) and less than 40 mmHg (e.g., less than 20, 10, 5, or 3 mmHg) partial pressure of any of the gases nitrogen, argon and helium.

In some embodiments, the head space gas phase comprises sulfur hexafluoride with a partial pressure above 10 mmHg and less than 3 mmHg partial pressure of any of the gases nitrogen, argon and helium, sulfur hexafluoride with a partial pressure above 20 mmHg and less than 5 mmHg partial pressure of any of the gases nitrogen, argon and helium, sulfur hexafluoride with a partial pressure above 50 mmHg and less than 10 mmHg partial pressure of any of the gases nitrogen, argon and helium, sulfur hexafluoride with a partial pressure above 100 mmHg and less than 20 mmHg partial pressure of any of the gases nitrogen, argon and helium, sulfur hexafluoride with a partial pressure above 200 mmHg and less than 40 mmHg partial pressure of any of the gases nitrogen, argon and helium, sulfur hexafluoride with a partial pressure above 500 mmHg and less than 40 mmHg partial pressure of any of the gases nitrogen, argon and helium, or sulfur hexafluoride with a partial pressure around 760 mmHg and less than 40 mmHg partial pressure of any of the gases nitrogen, argon and helium.

### III. Pharmaceutical Proteins and Formulations Thereof

As described herein, embodiments of the present invention provide stabilized pharmaceutical proteins (e.g., monoclonal antibodies; antibody-drug conjugates) and/or biological drugs.

A biological drug is any medicinal product manufactured in or extracted from biological sources. Most biological drugs are proteins or protein derivatives, however, the term also include sugars, nucleic acids, complex combinations and living entities such as cells and tissues. Typical examples of protein-based biological drugs include insulins, therapeutic enzymes, cytokines, interleukines, tumor necrosis factor, growth factors, therapeutic hormones and immunoglobulins including monoclonal antibodies and antibody-drug conjugates.

There are currently more than 100 different therapeutic proteins or protein derivatives in clinical use. In addition, very many new biological drugs are in development; especially within the field of monoclonal antibodies and antibody-drug conjugates. In addition, several biosimilar products are currently available and many biosimilars are in development.

A biosimilar product is a follow-on biological product developed by another producer than the originator. The drug substance in a biosimilar product is the same as the original product, the amino acid sequence is the same but since the process is somewhat different for a biosimilar product compared to the originator, there might be minor differences in the product. In biochemical processes producing complex biological proteins, the product is the process. Since there might be clinically relevant differences between two biological products produced be two different biological processes, the terminology “biosimilar” and not “generic” is used.

Protein drugs according to the present invention include any pharmacologically active protein or protein derivative to be used for parenteral delivery for diagnosis or treatment, including prophylactic treatment, of disease in humans and animals.

The protein can be a natural protein present in living organisms or a synthetic and/or semisynthetic protein.

The protein may be linked to another protein (e.g., by a linker). The protein may be linked to another non-protein (e.g., small molecule (e.g., drug), nucleic acid, sugar, etc.).

The protein can be any protein for therapeutic or diagnostic use. The proteins can for example be adrenocortitropin hormones like cyctokines like anakinra, consensus interferon, interferons (alpha, beta, gamma), Interleukins (type 1, 2 or 11), enzymes like for example imiglucerase and idursulfase, gonadotropins like for example flooitropin (alpha or beta), lutropin alfa, menotropin, gonadotropin-releasing hormones like abarelix, cetrorelix acetate, goserelin acetate, leuprolide acetate, growth hormones like somatropin and pegvisomat, hematrooietic growth factors like erythropoietins, filgrastim, pegfilgrastinm, pancreatic hormones like insulin and insulin analogs, adrenocorticotropin hormone, and cosyntropin, corticotropin-releasing factor like corticorelin ovine triflutate, parathyroid hormones, pituitary hormones, placental hormones and monoclonal antibodies.

Monoclonal antibodies include any monoclonal antibody or fragments or derivatives thereof. Some examples of antibodies include bagovomab, abciximab, actoxumab, adalimumab, adecatumumab, aducanumab, afelimomab, afutuzumab, alacizumab pegol, alemtuzumab, alirocumab, altumomab pentetate, amatuximab, anatumomab mafenatox, anifrolumab, anrukinzumab, apolizumab, arcitumomab, aselizumab, atinumab, atlizumab, atorolimumab, bapineuzumab, basiliximab, bavituximab, bectumomab, belimumab, benralizumab, bertilimumab, besilesomab, bevacizumab, bezlotoxumab, biciromab, bimagrumab, bivatuzumab mertansine, blinatumomab, blosozumab, brentuximab vedotin, briakinumab, brodalumab, canakinumab, cantuzumab mertansine, cantuzumab ravtansine, caplacizumab, capromab pendetide, carlumab, catumaxomab, cedelizumab certolizumab pegol, cetuximab, citatuzumab bogatox, cixutumumab, clazakizumab, clenoliximab, clivatuzumab tetraxetan, conatumumab, concizumab, crenezumab, dacetuzumab, daclizumab, dalotuzumab, daratumumab, demcizumab, denosumab, detumomab, dorlimomab aritox, drozitumab, duligotumab, dusigitumab, ecromeximab, eculizumab, edobacomab, falizumab, efungumab, eldelumab, elotuzumab, elsilimomab, enavatuzumab, enlimomab pegol, enokizumab, enoticumab, ensituximab, epitumomab cituxetan, epratuzumab, erlizumab, ertumaxomab, etaracizumab, abegrinmab, etrolizumab, evolocumab, exbivirumab, faralimomab, farletuzumab, fasinumab, felvizumab, fezakinumab ficlatuzumab, figitumumab, flanvotumab, fontolizumab, foralumab, Foravirumab, fresolimumab, fulranumab, futuximab, galiximab, ganitumab, gantenerumab, gavilimomab, gemtuzumab, gevokizumab, girentuximab, glembatumumab vedotin, golimumab, gomiliximab, guselkumab, Ibalizumab, ibritumomab tiuxetan, icrucumab, igovomab, imciromab, imgatuzumab, inclacumab, indatuximab, iravtansine, infliximab, intetumumab, inolimomab, minotuzumab ozogamicin, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, ligelizumab, lintuzumab, lirilumab, lodelcizumab, lorvotuzumab mertansine, lucatumumab, lumiliximab, mapatumumab, margetuximab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-CD3, nacolomab tafenatox, namilumab, naptumomab estafenatox[, narnatumab, natalizumab, nebacumab, necitumumab, nerelimomab, nesvacumab, nimotuzumab, nivolumab bofetumomab merpentan, ocaratuzumab, ocrelizumab, odulimomab, ofatumumab olaratumab, olokizumab, omalizumab, onartuzumab, ontuxizumab, oportuzumab monatox, oregovomab, orticumab, otelixizumab, otlertuzumab, oxelumab, ozanezumab, ozoralizumab, pagibaximab, palivizumab, panitumumab, pankomab, panobacumab, parsatuzumab, pascolizumab, pateclizumab, patritumab, pemtumomab, perakizumab, pertuzumab, pexelizumab, pidilizumab, pinatuzumab vedotin, pintumomab, placulumab, polatuzumab vedotin, ponezumab, priliximab, pritoxaximab, pritumumab, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, seribantumab, setoxaximab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, teneliximab, teplizumab, teprotumumab, ticilimumab tildrakizumab, tigatuzumab, tocilizumab, toralizumab, tositumomab, tovetumab, tralokinumab, trastuzumab, tregalizumab, tremelimumab, tucotuzumab, celmoleukin, tuvirumab, ublituximab, urelumab, urtoxazumab, ustekinumab, vantictumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox.

Antibody-drug conjugates are molecules comprising an antibody (e.g., a whole antibody (e.g., a whole monoclonal antibody)) or an antibody fragment (e.g., a single-chain variable fragment, minibody, diabody, etc.) linked by a stable chemical linker to a biologically active drug (e.g., a cytotoxic (e.g., anticancer) agent or drug). In some embodiments, the linker comprises at least one bond that is labile under some biological conditions (e.g., when in the intracellular space). When administered, an antibody-drug conjugate binds to its target antigen. Then, in some embodiments the antibody-drug conjugate is internalized through receptor-mediated endocytosis. This facilitates the subsequent release of the drug (e.g., cytotoxin), e.g., by breaking the labile bond. The drug then provides its biological effects at the targeted site, e.g., for cancer drugs, the deployment of the drug (e.g., cytotoxin) produces apoptotic cell death of the cancer cell. The three components of antibody-drug conjugates (e.g., the antibody (e.g., monoclonal antibody), linker, and drug (e.g., cytotoxin)) provide for controlling the targeting, efficacy, and toxicity of the antibody-drug conjugate. See, e.g., Peters and Brown (2015) “Antibody-drug conjugates as novel anti-cancer chemotherapeutics” Bioscience Reports 35, e00225, doi:10.1042/BSR20150089.

Antibody-drug conjugates include any antibody-drug conjugate or fragments or derivatives thereof. Some examples of antibody-drug conjugates include trastuzumab emtansine, gemtuzumab ozogamicin, and brentuximab vedotin. However, the technology is not limited to these particular antibody-drug conjugates and is applicable to any antibody-drug conjugate that is extant, currently in development or testing, or that is yet-to-be-developed. Other examples of antibody-drug conjugates include, e.g., T-DM1, Inotuzumab ozogamicin, Pinatuzumab vedotin, RG-7596, Lifastuzumab vedotin, Glembatumumab vedotin, Coltuximab Ravtansine (SAR-3419), Lorvotuzumab mertansine (IMGN-901), Indatuximab Ravtansine (BT-062), Anti-PSMA ADC, Labetuzumab-SN-38, MLN-0264, ABT-414, and Milatuzumab doxorubicin.

In some embodiments, the present invention provides stabilization agents or methods as described above and other additives well known additives for protein-based pharmaceutical formulations described in the scientific literature, patent literature and present in commercial products.

Some of these preferred additives include, for example, sugars like trehalose, sucrose, maltose, fructose, raffinose, lactose and glucose, surfactants like for example polysorbate 20, polysorbate 40, Polysorbate 80, Polyoxamer 188, Polyoxamer 407, polyols like glycerol, mannitol, sorbitol, xylitol and cyclodextrins, polymers like polyethylene glycol, dextran, polyvinylpyrrolidone, chelating agents like EDTA and DTPA, antioxidants like ascorbic acid and glutathione, preservatives like benzyl alcohol, m-cresol, methionine, citric acid and various ions and buffers within the scope and limitations in the present document.

The pH of the formulation can vary from 3 to 9, most formulations have a pH between 4.5 and 7.4. Most formulations of antibodies have, according to the present invention a pH between 5.5 and 7.2.

If the drug formulation is in the form of an aqueous solution, the formulation might be for direct administration to the patient. The preferred administration routes are parenteral administration, however, some biological drugs might be administered orally. The most preferred administration route is intravascular administration, subcutaneous administration and administration through nose and lungs. The even more preferred administration routes are intravenous administration and subcutaneous administration.

The formulation might also be in the form of a concentrate for dilution before use. The concentration of the biological drug in the formulation will vary, depending upon several factors: choice of biological drug, efficacy of the drug, safety of the drug, clinical indication and administration route. Typical concentration range is from 0.01 mg to 300 mg per ml. The stability problem with formation of dimers, oligomers, aggregates or precipitates is often a larger problem if the protein concentration is high. If the formulation is meant for subcutaneous administration the injection volume is limited (typically less than 1 ml) so such formulations have generally a high concentration of the protein.

One further aspect of the present invention is therefore related to new protein formulations for subcutaneous characterized by the presence of one or more of the stabilization agents described herein.

In some embodiments, the formulations are concentrates for dilution before use.

In some embodiments, compositions comprise formulations for subcutaneous administration comprising 30 to 300 mg protein (e.g., monoclonal antibody) per ml (e.g., 70 to 200 mg proteins per ml).

The clinical dose of the biological drug in the formulation will vary, depending upon several factors: choice of biological drug, efficacy of the drug, safety of the drug, clinical indication and administration route. Typical clinical doses are from 0.001 mg to 1.00 gram protein.

The osmolality of the present aqueous formulations or formulations prepared by dissolving dry material prior to use, can vary. If the formulation is a concentrate to be diluted before administration, the osmolality is generally not relevant per se, since the osmolality then typically will be the osmolality of the dilution media. The osmolality of the final solution administered to the patients should in this case preferably be around 300 mOsm/kg, which is the osmolality of blood, if the injection volume/infusion volume is high.

If the formulation per se or after dissolution of dry material is intended for direct administration to patients, the osmolality should be between 200 mOsm/kg and 3000 mOsm/kg, preferably between 250 mOsm/kg and 2000 mOsm/kg, most preferably around 300 mOsm/kg (isotonic with blood) or light hypertonic (300-900 mOsm/kg).

In some embodiments, formulations for parenteral administration, according to the present invention, are sterile formulations. The present formulations can be sterilized using the same sterilization techniques used for sterilization of pharmaceutical formulations comprising proteins. The most useful methods include sterile filtration.

The formulation can be filled in vials or devices for single use or for multiple uses.

If the protein is in the form of a powder, the powder might be filled into vials or devices optionally together with additives, or the protein optionally with additives might be filed into vials or devices as an aqueous solution followed by drying. A preferred drying method is freeze drying. This process is preferably performed as an aseptic production process with sterile vial or devices and sterile equipment.

The vials can typically be 0.5-10 ml borosilicate glass vials with elastomer stopper (rubber, teflon) and a seal (aluminium crimp overseal).

Typical devices could be prefilled syringes for injections, dry injectors and inhalation devises.

If the protein is in the form of a powder, the product might be a kit comprising two units, one unit comprising the dry material (protein and optionally additives) and one unit comprising water and optionally additives. The additives present in the dry powder comprising protein might vary and will typically comprise additives that stabilize the dry protein powder. These additives might for example be a cryprotective and/or lyoprotective agents like for example sucrose or trehalose. The mesylate comprising additives according to the present invention might be present in the dry protein powder, in the aqueous solution or in both the dry powder and the aqueous solution.

One aspect of the present invention relates to production processes for proteins where stabilization agents described herein are present to stabilize the protein. This production process includes all steps in production, process control, purification and quality control of the protein-based drug substance and protein-based drug product. This include for example production of relevant cell line, the bioreactor production process (upstream), the various steps in the isolation and purification process including various chromatographic process steps (downstream process) and quality control steps using different analytic techniques including HPLC methods.

The methods used for stability testing can be all state of the art methods for analysis of proteins. These methods are described in various text books, scientific publications and patent documents related to protein drug formulations. Some of the most useful methods for analysis of stability of the new formulations here includes: size exclusion chromatography, especially size exclusion high performance chromatography (SEC-HPLC), turbidity measurements, electrophoresis methods, infrared spectroscopy especially Fourier-Transform infrared spectroscopy (FT-IR), UV spectroscopy, florescence spectroscopy, light-scattering techniques and calorimetry.

### EXAMPLES

The invention is further illustrated by the following non-limiting examples:

### Example 1

Preparation of Meglumine Mesylate Salt

N-methylglucamine (19.5 g, 100 mmol) and methanesulphonic acid (9.6 g, 100 mmol) were dissolved in water (50 ml). The mixture was stirred at room temperature for 1 hour and evaporated. Meglumine mesylate salt was isolated as clear viscous oil (28.5 gram)

### Example 2

Preparation of Histidine Mesylate Salt

L-histidine (15.5 g, 100 mmol) and methanesulphonic acid (9.6 g, 100 mmol) were dissolved in water (100 ml). The mixture was stirred at room temperature for 1 hour and evaporated. L-histidine mesylate salt was isolated as white crystalline material (28.5 gram)

### Example 3

Formulation of canakinumab 150 mg/ml (freeze dried)

| | |
|---|---|
| Canakinumab | 120 g |
| Sucrose | 92.4 g |
| L-histidine and L-histidine mesylate | 1.0 g |
| Polysorbate 80 | 0.72 g |

The components are dissolved in water (1000 ml) under stirring. The solution (1200 ml) is sterile filtered and 1.2 ml of the solution is filled in borosilicate glass vials (2 ml). The vials are freeze dried and closed (rubber stopper and aluminium crimp seal). The vials are labelled according to relevant EU Directive. Another vial comprises 1.5 ml sterile water. The vials are packed together with a packet insert.

The packet insert instruct the user to add 1.2 ml sterile water to the vial with dry powder using a syringe, swirl the vial slowly at an angle of about 45° for approximately 1 minute and allow to stand for 5 minutes. The vial must not be shaken. Then, the vial should be turned upside down and back again ten times. Administer 1.0 ml to the patient subcutaneously with a syringe.

### Reference Example

Ilaris® from Novartis. The Ilaris® formulation comprise L-histidine HCl salt and not L-histidine mesylate salt.

ILARIS (canakinumab) is an interleukin-1β blocker indicated for the treatment of Cryopyrin-Associated Periodic Syndromes (CAPS), in adults and children 4 years of age and older including: Familial Cold Autoinflammatory Syndrome (FCAS), Muckle-Wells Syndrome (MWS) and for treatment of active Systemic Juvenile Idiopathic Arthritis (SJIA) in patients aged 2 years and older.

### Example 4

Formulation of golimumab 100 mg/ml (solution)

| | |
|---|---|
| Golimunmab | 120 g |
| Sorbitol | 92.4 g |
| L-histidine and L-histidine mesylate | 1.0 g |
| Polysorbate 20 | 2.2 g |

The components are dissolved in water (1000 ml) under stirring. The solution is sterile filtered and 1.2 ml of the solution is filled in borosilicate glass vials (2 ml) and closed (rubber stopper and aluminium crimp seal). 1.0 ml comprises 100 mg golimumab. The vials are labelled according to relevant EU Directive.

### Example 5

Simponi® from Janssen Biologics. The Simponi formulation comprise L-histidine HCl salt and not L-histidine mesylate salt. It is in the form of a pre-filled pen. Simponi® (Golimumab) is a tumour necrosis factor alpha (TNF-α) inhibitor for treatment of rheumatoid arthritis (RA), psoriatic arthritis (PsA), ankylosing spondylitis (AS) and Ulcerative colitis (UC).

### Example 6

Preparation of Meglumine Citrate Salt

N-methylglucamine (17.77 g, 90 mmol) and citric acid monohydrate (6.3 g, 30 mmol) were dissolved in water (100 ml). The mixture was stirred at room temperature for 1 hour and evaporated. Meglumine citrate salt was isolated as a white solid material (23.5 gram)

### Example 7

Formulation of alefacept 15 mg/ml (lyophilized)

| | |
|---|---|
| Alefacept | 15.0 g |
| Glycine | 10.0 g |
| Meglumine citrate | 30.0 g |
| Citric acid | to pH 6.9 |

The components are dissolved in water (1000 ml) under stirring. The solution is sterile filtered and 1.2 ml of the solution is filled in borosilicate glass vials (2 ml). The vials are freeze dried and closed (rubber stopper and aluminium crimp seal). The vials are labelled according to relevant EU Directive. Another vial comprises 1.5 ml sterile water. The vials are packed together with a packet insert.

The packet insert instruct the user to add 1.2 ml sterile water to the vial with dry powder using a syringe, swirl the vial slowly at an angle of about 45° for approximately 1 minute and allow to stand for 5 minutes. The vial must not be shaken. Then, the vial should be turned upside down and back again ten times. Administer 1.0 ml to the patient subcutaneously with a syringe.

### Reference Example

Amevive® from Astellas Pharma. The Ilaris® Formulation Comprises Sodium Citrate and not Meglumine Citate.

Amevive® (alefacept) is an immunosuppressive dimeric fusion protein that consists of the extracellular CD2-binding portion of the human leukocyte function antigen-3 (LFA-3) linked to the Fc (hinge, CH2 and CH3 domains) portion of human IgG1. Alefacept is produced by recombinant DNA technology in a Chinese Hamster Ovary (CHO) mammalian cell expression system. The molecular weight of alefacept is 91.4 kilodaltons. Amevive® is indicated for the management of patients with moderate to severe chronic plaque psoriasis in adult patients.

### Example 8

Formulation of adalimubab (50 mg/ml)

| | |
|---|---|
| Adalimumab | 50.0 g |
| Meglumine mesylate | 20.0 g |
| Meglumine citrate | 0.86 g |
| Polysorbate 80 | 1.0 g |
| Mannitol | 12.0 g |
| Citric acid | to pH 5.2 |

The components are dissolved in water (1000 ml) under stirring. The solution is sterile filtered and 1.2 ml of the solution is filled in borosilicate glass vials (2 ml).

1 ml comprises 50 mg adalimumab.

### Reference Example

Humira® is from AbbVie. The Humira® formulation comprises sodium chloride and sodium citrate and not meglumine mesylate and meglumine citrate.
Humira® (adalimumab) is a TNF inhibiting anti-inflammatory drug. Humira® is used in the treatment of several conditions where the suppression of the immune response is desired. The molecular weight of adalimumab is 144190 D.

### Example 9

Etanercept Injection Formulation with Low Partial Pressure of Nitrogen in Head Space

Etanercept is a fusion (chimeric) protein produced by recombinant DNA. The protein inhibits tumor necrosis factor (TNF/TNF-alpha) which is a soluble inflammatory cytokine. The protein is a complex molecule with a molecular weigh of appr. 15 000 Da. The original etanercept product is Enbrel which is marketed in Norway by Pfizer. The indications for Enprel include moderate to severe rheumatoid arthritis and psoriatic arthritis.

The formulation is prepared by dissolving etanercept, sucrose, sodium chloride, L-arginine hydrochloride, sodium dihydrogen phosphate monohydrate and disodium hydrogen phosphate in water under aseptic condition followed by sterile filtration and filling into vials under nitrogen. The vials are closed under vacuum.

The concentrations of each ingredient are:

| | |
|---|---|
| Etanercept | 50 mg/ml |
| Sucrose | 10 mg/ml |
| Sodium chloride | 5.5 mg/ml |
| L-arginine hydrochloride | 2.6 mg/ml |
| Sodium dihydrogen phosphate monohydrate | 0.9 mg/ml |
| Disodium hydrogen phosphate | to pH 6.2 |
| Water | |

The total pressure in head space is 100 mmHg.
Air is added to the vial by injection to establish appr. atmospheric pressure before the solution in withdrawn from the vial with a syringe.

### Example 10

Rituximab Injection Formulation with Perfluoropropane in Head Space

Trituximab is chimeric monoclonal antibody against the protein CD20 which is primarily found on the surface of B cells which comprise a part of the immune system. The molecular weight is appr. 144.000 Da. The original rituximab product is MabThera which is marketed in Norway by Roche. The indications for MabThera include non-Hodgkins lymphoma, chronic lymphatic leukemia and rheumatoid arthritis.

The formulation is prepared by dissolving rituximab, sodium chloride, sodium cirate dihydrate, polysorbate 80 in water under aseptic condition followed by sterile filtration and filling into vials under nitrogen. The vials are closed under perfluoropropane gas.

The concentrations of each ingredient are:

| | |
|---|---|
| Rituximab | 10 mg/ml |
| Sodium chloride | 9 mg/ml |
| Sodium cirate dihydrate | 7.35 mg/ml |
| Polysorbate 80 | 0.7 mg/ml |

### Example 11

Trastuzumab Aqueous Solution with Carbon Dioxide in Head Space.

Trastuzumab is a monoclonal antibody with affinity for HER2 receptor. The molecular weight is appr. 146.000 Da. The original rituximab product is Herceptin which is marketed in Norway by Roche. The main indication for Herceptin is HER2-positive breast cancer.

The formulation is prepared by dissolving trastuzumab, recombinant human hyaluronidase, L-histidine hydrochloride monohydrate, L-histidine, trehalose, L-methionine, polysorbate 20, sodium cirate dihydrate, polysorbate 80 in water under aseptic condition followed by sterile filtration and filling into vials under carbon dioxide. The vials are closed under carbon dioxide gas. The concentration of trastuzumab is 120 mg/ml.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

1-99. (canceled)

100. A composition, comprising a polypeptide and at least one methanesulfonic acid or salt thereof.

101. The composition of claim 100, wherein said polypeptide is a water soluble protein.

102. The composition of claim 100, wherein said polypeptide is a pharmaceutical polypeptide.

103. The composition of claim 102, wherein said pharmaceutical polypeptide is a monoclonal antibody or an antibody-drug conjugate.

104. The composition of claim 100, wherein said composition further comprises an aqueous liquid.

105. The composition of claim 104, wherein said aqueous liquid is a pharmaceutically acceptable carrier.

106. The composition of claim 100, wherein said composition is a dry formulation.

107. The composition of claim 100, wherein said methanesulfonic acid or salt thereof is a pharmacologically inactive salt of methanesulfonic acid.

108. The composition of claim 100, wherein said polypeptide is more stable that in the absence of said methanesulfonic acid or salt thereof.

109. The composition of claim 100, wherein said polypeptide exhibits reduced formation of dimers, polymers, aggregates and/or precipitates relative to the level in the absence of said methanesulfonic acid or salt thereof.

110. A method of stabilizing a polypeptide, comprising, contacting the polypeptide with a composition comprising at least one methanesulfonic acid or salt thereof.

111. The method of claim 110, wherein said polypeptide is a water soluble protein.

112. The method of claim 110, wherein said polypeptide is a pharmaceutical polypeptide.

113. The method of claim 112, wherein said pharmaceutical polypeptide is a monoclonal antibody or an antibody-drug conjugate.

114. The method of claim 110, wherein said polypeptide exhibits reduced formation of dimers, polymers, aggregates and/or precipitates relative to the level in the absence of methanesulfonic acid.
